(19) United States
(12) Patent Application Publication
(10) Pub. No.: US 2015/0282879 A1
(43) Pub. Date: Oct. 8, 2015

(54) PRECISE INSERTION SITE LOCATOR

(71) Applicant: MEDTRONIC MINIMED, INC., Northridge, CA (US)

(72) Inventor: Jose J. Ruelas, San Fernando, CA (US)

(73) Assignee: MEDTRONIC MINIMED, INC., Northridge, CA (US)

(21) Appl. No.: 14/244,094

(22) Filed: Apr. 3, 2014

(51) Int. Cl. A61B 19/00; A61B 5/00

(52) U.S. Cl. CPC A61B 19/201; A61B 5/6847; A61B 5/6833; A61B 2562/226; A61B 2562/08; A61B 2562/12

(57) ABSTRACT

An insertion system to precisely locate the insertion site for a sensor is disclosed. The insertion system includes an insertion guide with a protective layer, a guide layer and a liner layer. The protective layer is on top of the guide layer and the guide layer has a first locating structure and adhesive on the bottom. The liner layer is below the guide layer. The insertion system further includes an insertion tool with a corresponding first locating structure that interfaces with the first locating structure, wherein alignment of the first locating structure and corresponding first locating structure establishes placement of the insertion tool and subsequently the sensor.

### FIELD OF THE INVENTION

This invention relates to monitor systems and, in particular embodiments, to devices and methods for insertion of a sensor to determine a characteristic of a body.

### BACKGROUND OF THE INVENTION

Over the years, bodily characteristics have been determined by obtaining a sample of bodily fluid. For example, diabetics often test for blood glucose levels. Traditional blood glucose determinations have utilized a painful finger prick using a lancet to withdraw a small blood sample. This results in discomfort from the lancet as it contacts nerves in the subcutaneous tissue. The pain of lancing and the cumulative discomfort from multiple needle pricks is a strong reason why patients fail to comply with a medical testing regimen used to determine a change in characteristic over a period of time. Although non-invasive systems have been proposed, or are in development, none to date have been commercialized that are effective and provide accurate results. In addition, all of these systems are designed to provide data at discrete points and do not provide continuous data to show the variations in the characteristic between testing times.

A variety of implantable electrochemical sensors have been developed for detecting and/or quantifying specific agents or compositions in a patient's blood. For instance, glucose sensors have been developed for use in obtaining an indication of blood glucose levels in a diabetic patient. Such readings are useful in monitoring and/or adjusting a treatment regimen which typically includes the regular administration of insulin to the patient. Thus, blood glucose readings improve medical therapies with semi-automated medication infusion pumps of the external type, as generally described in U.S. Pat. Nos. 4,562,751; 4,678,408; and 4,685,903; or automated implantable medication infusion pumps, as generally described in U.S. Pat. No. 4,573,994, which are herein incorporated by reference. Typical thin film sensors are described in commonly assigned U.S. Pat. Nos. 5,390,671; 5,391,250; 5,482,473; and 5,586,553 which are incorporated by reference herein, also see U.S. Pat. No. 5,299,571. However, the monitors for these continuous sensors provide alarms, updates, trend information and require sophisticated hardware to allow the user to program the monitor, calibrate the sensor, enter data and view data in the monitor and to provide real-time feedback to the user. This sophisticated hardware makes it most practical for users that require continuous monitoring with feedback to maintain tight control over their conditions. In addition, these systems require the user to be trained in their use, even if to be worn for short periods of time to collect medical data which will be analyzed later by a doctor.

Doctors often need continuous measurements of a body parameter over a period of time to make an accurate diagnosis of a condition. For instance, Holter monitor systems are used to measure the EKG of a patient's heart over a period of time to detect abnormalities in the heart beat of the patient. Abnormalities detected in this manner may detect heart disease that would otherwise go undetected. These tests, while very useful are limited to monitoring of bio-mechanical physical changes in the body, such as a heart beat, respiration rate, blood pressure or the like.

Placement on the body and insertion depth of an electrochemical sensor can be very important. As many electrochemical sensors are designed to work in a specific type of bodily fluid placing a sensor intended for use in interstitial fluid within a blood vessel will not provide the desired data Likewise, there may be preferred location on the body to place a sensor that minimizes the likelihood of the sensor being dislodged from a preferred depth/location. Many insertion aid devices or “serters” can provide a level of accuracy for the insertion site. However, such serters can only provide accurate placement and not precise placement. A precise insertion site locator component is needed to help give serters accuracy and precision.

### SUMMARY OF THE DISCLOSURE

An insertion system to precisely locate the insertion site for a sensor is disclosed. The insertion system includes an insertion guide with a protective layer, a guide layer and a liner layer. The protective layer is on top of the guide layer and the guide layer has a first locating structure and adhesive on the bottom. The liner layer is below the guide layer. The insertion system further includes an insertion tool with a corresponding first locating structure that interfaces with the first locating structure, wherein alignment of the first locating structure and corresponding first locating structure establishes placement of the insertion tool and subsequently the sensor.

Other features and advantages of the invention will become apparent from the following detailed description, taken in conjunction with the accompanying drawings which illustrate, by way of example, various features of embodiments of the invention.

### BRIEF DESCRIPTION OF THE DRAWINGS

A detailed description of embodiments of the invention will be made with reference to the accompanying drawings, wherein like numerals designate corresponding parts in the several figures.

FIGS. 1A-1D are exemplary illustrations of components of an insertion system to precisely locate an insertion site for a sensor, in accordance with one embodiments of the present invention.

FIGS. 2A-2D are exemplary illustrations of using a guide tool with a modified footprint to precisely location an insertion site, in accordance with one embodiment of the present invention.

FIGS. 3A and 3B are exemplary illustrations of multiple locating structures on an insertion system to precisely locate an insertion site for a sensor, in accordance with one embodiments of the present invention.

FIGS. 4A-4C are exemplary illustrations an insertion system that includes a guide tool being used in conjunction with multiple locating structures to precisely locate an insertion site for a sensor, in accordance with one embodiment of the present invention.

FIGS. 5A and 5B further illustration another embodiment where a first locating structure is used in conjunction with a guide layer and the insertion tool to create a cohesive structure, in accordance with one embodiment of the present invention.

FIGS. 6A-6G illustrate various operations that enable use of the insertion guide, in accordance with one embodiment of the present invention.

### DETAILED DESCRIPTION

As shown in the drawings for purposes of illustration, one embodiment of the invention is as an insertion guide and insertion tool for a sensor and/or cannula. Selecting an exact location for introducing a transcutaneous sensor and/or cannula is important for some patient and clinicians because there are often reasons why a specific location must be targeted. A precise insertion site guide can include a template with a locating window and targets for the insertion site. In some embodiments a clear or translucent film with targets over the locating window will allow a patient or clinician to see the exact location of insertion. Many embodiments of the insertion guide utilize repositionable skin friendly adhesives that allow the user to place the insertion guide on different locations without causing pain or discomfort. The clear window or protective layer is removed when the desired insertion site is located. The insertion tool is then placed within the guide and insertion of the sensor, cannula and/or both is completed.

In some embodiments once the sensor is installed the sensor data is recorded into memory integrated into an electronics package that also provides power and wireless communication capability to the sensor. In other embodiments the sensor transmits sensor readings to an infusion pump that can include memory to store the sensor readings. The recorded sensor readings or data can later be downloaded or transferred to a computing device to determine body characteristic data based on the data recording over the period of time. In embodiments of the present invention, the analyte sensor set and monitor system are for determining glucose levels in the blood and/or bodily fluids of the user without the use of, or necessity of, complicated monitoring systems that require user training and interaction. However, it will be recognized that further embodiments of the invention may be used to determine the levels of other analytes or agents, characteristics or compositions, such as hormones, cholesterol, medications concentrations, viral loads (e.g., HIV), or the like. The monitor system and analyte sensor are primarily adapted for use in subcutaneous human tissue. However, still further embodiments may be placed in other types of tissue, such as muscle, lymph, organ tissue, veins, arteries or the like, and used in animal tissue. The analyte sensors may be subcutaneous sensors, transcutaneous sensors, percutaneous sensors, sub-dermal sensors, skin surface sensors, or the like. Embodiments may measure and record sensor readings on an intermittent or continuous basis. For purposes of this disclosure the term “bottom” should be construed to be closer to being in contact with a patient's skin. Additionally, the term “top” is defined as the opposite side of “bottom”, or a side opposite that which is contact or closer to a patient's skin.

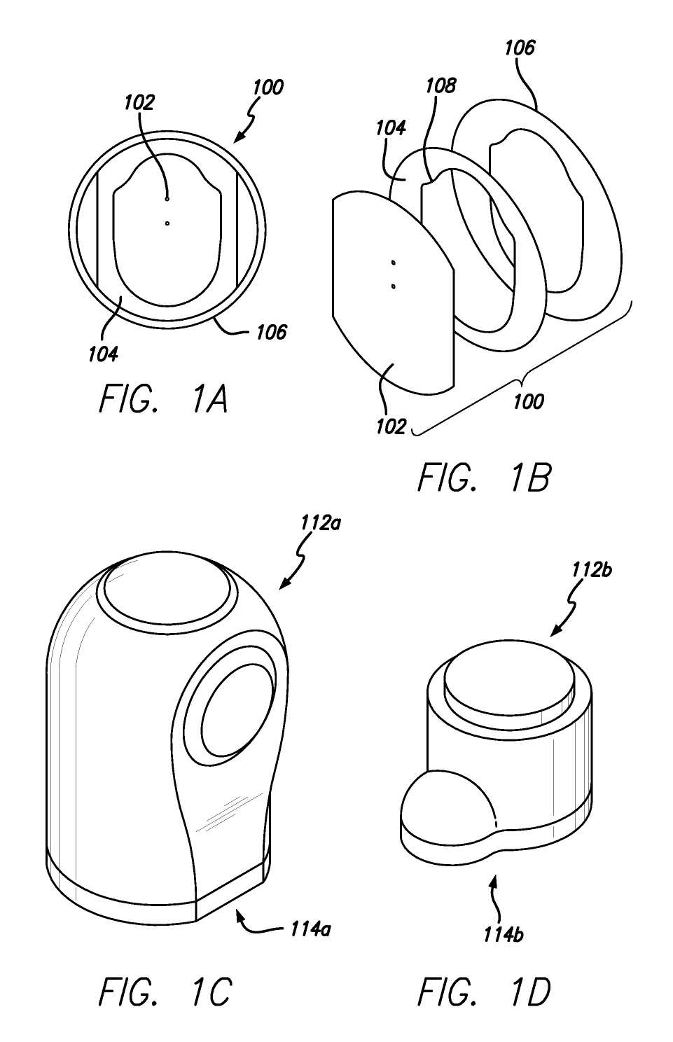

FIGS. 1A-1D are exemplary illustrations of components of an insertion system to precisely locate an insertion site for a sensor, in accordance with one embodiments of the present invention. The insertion system includes an insertion guide 100, seen in FIGS. 1A and 1B and an insertion tool similar to exemplary insertions tools 112a and 112b in FIGS. 1C and 1D. In some embodiments the insertion guide 100 is composed of multiple layers. In the embodiment illustrated in FIGS. 1A and 1B the insertion guide 100 is made up of three layers. There is a protective layer 102, a guide layer 104 and a liner layer 106. The protective layer 102 can be transparent or translucent allowing visual inspection of the guide layer 104 and some aspects of the liner layer 106. The guide layer 104 includes first locating structure 108. The guide layer 104 includes a locating/guide layer interface and a guide/liner layer interface. The guide/liner layer interface includes a medical grade repositionable adhesive on the bottom of guide layer 104. In some embodiments, the liner layer 106 is light card stock. The finish of the liner layer 106 that interfaces with the guide layer 104 can be a glossy non-stick finish that easily allows the guide layer 104 to be separated from the liner layer 106.

Two different types of insertion tools 112a and 112b are shown in FIGS. 1C and 1D each having a respective footprint 114a and 114b. The footprints 114a and 114b of each insertion tool 112a and 112b is defined as the shape traced around the bottom of the insertion tool. In some embodiments the footprint of the insertion tool and the first locating structure 108 of the guide layer are identical. The exemplary insertion tools shown in FIGS. 1C and 1D should not be construed to be limiting. Other insertion tools can be used with various footprints, sizes, and operations. The sensor is not shown in FIGS. 1A-1D because the sensor is not critical to understanding the various components of the insertion system to precisely locate an insertion site for a sensor. In one embodiment the sensor is a glucose sensor that is intended to be implanted in the subcutaneous tissue of a patient. In other embodiments, the sensor can be placed within a blood vessel, percutaneously, or other various insertion locations, depths or techniques.

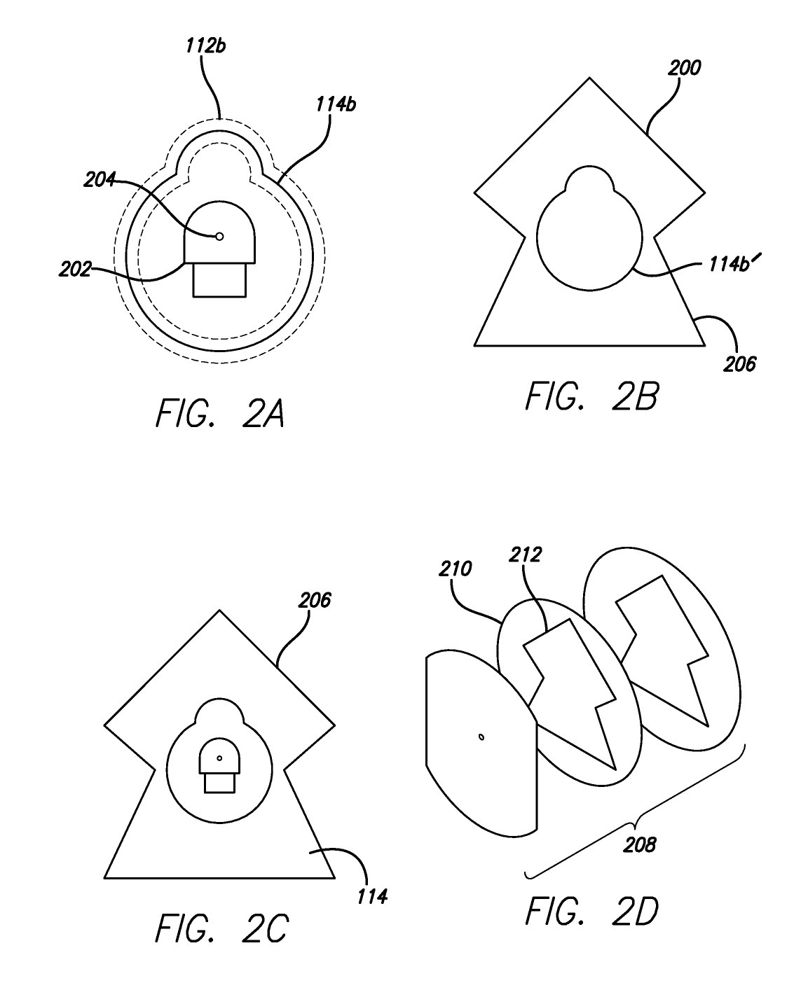

FIGS. 2A-2D are exemplary illustrations of using a guide tool 200 with a modified footprint 114b′ to precisely location an insertion site, in accordance with one embodiment of the present invention. FIG. 2A is a view from the bottom of the insertion tool 112b. The footprint 114b of the insertion tool 112b is visible between the dotted lines. For illustrative purposes, an exemplary sensor 202 is shown roughly centered within the insertion tool 112. As the sensor 202 is being viewed from the bottom, the sensor 204 appears as a circle, or dot.

FIG. 2B shows a guide tool 200 that is configured to be removeably coupled to the insertion tool 112b. In some embodiments the guide tool 200 removeably snaps into place with the footprint 114b of the insertion tool fitting within the footprint 114b′ within the guide tool 200. FIG. 2C is an illustration of the bottom of the insertion tool 112b after it has been installed within the guide tool 200. The guide tool 200 changes the footprint of the insertion tool 112b from 114b to a modified footprint 206. FIG. 2D is an illustration of the layers of an insertion guide 208 that is configured to work with the insertion tool 112b after it has been installed in guide tool 200. Guide layer 210 has first locating structure 212 defined to accommodate modified footprint 206.

The shape of modified footprint 206 should not be construed as limiting. Modified footprint 206 can be any shape that assists in the placement of the sensor. In some embodiments, the modified footprint 200 can be selected to be appealing to children, such as, but not limited to animal shapes, fruit shapes, cartoon characters, officially licensed toys and characters and the like. In some embodiments the guide tool 200 is reusable, while in other embodiments the guide tool 200 is intended to be disposable. In embodiments where the guide too 200 is intended to be reused, the material for the guide tool would be selected from those that are easily sterilized using common hospital or household techniques.

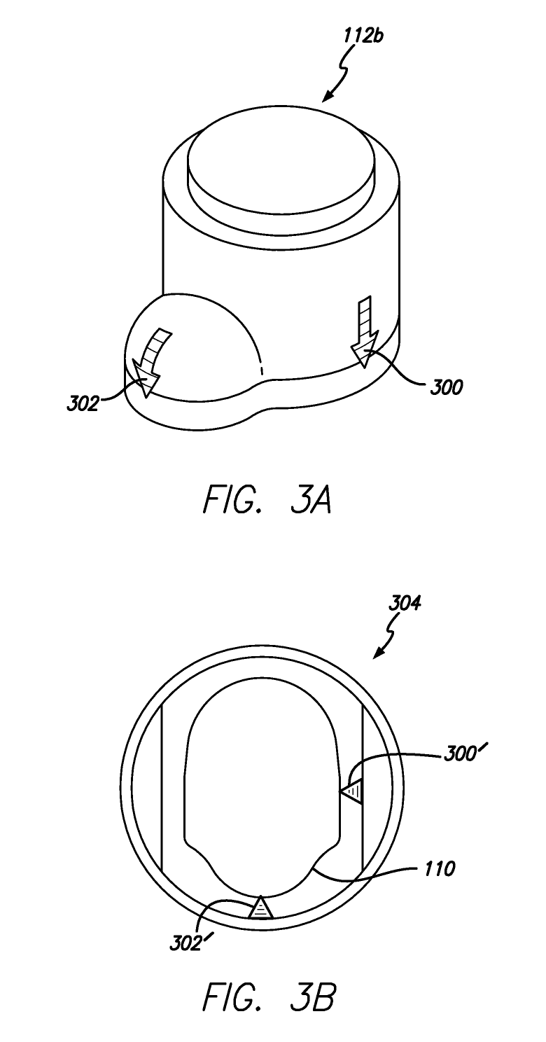

FIGS. 3A and 3B are exemplary illustrations of multiple locating structures on an insertion system to precisely locate an insertion site for a sensor, in accordance with one embodiments of the present invention. FIG. 3A is a perspective view of an insertion tool 112b that includes a second locating structure 300 and third locating structure 302. In some embodiments, second locating structure 300 is molded into the shape of the insertion tool 112b while third locating structure 302 is printed onto the insertion tool 112b. In other embodiments, both second and third locating structures 300 and 302 are molded into the insertion tool 112b. In still other embodiments both second and third locating structures 300 and 302 are printed onto the insertion tool 112b. FIG. 3B is a top view of an insertion guide 304 that is intended to be aligned with insertion tool 112b. Printed onto the guide layer is second corresponding locating structure 300′ and third corresponding locating structure 302′. The first locating structure 110 configured to follow the footprint of insertion tool 112b is also visible from the top view of the insertion guide 304 in FIG. 3B. Additional or fewer locating structures can be implemented in the embodiment shown in FIGS. 3A and 3B. Furthermore, the embodiments shown in FIGS. 3A and 3B should be considered exemplary and not limiting. For example, while second locating structure 300 and second corresponding locating structure 300′ are shown in black and white in FIGS. 3A and 3B various colors and patterns of printing could be used on both the insertion tool 112b and the guide layer to better correlate the relationship between the second locating structures. In still other embodiments, second corresponding locating structure 300′ and third corresponding locating structure 302′ can be die-cut from the foam just as first locating structure 110.

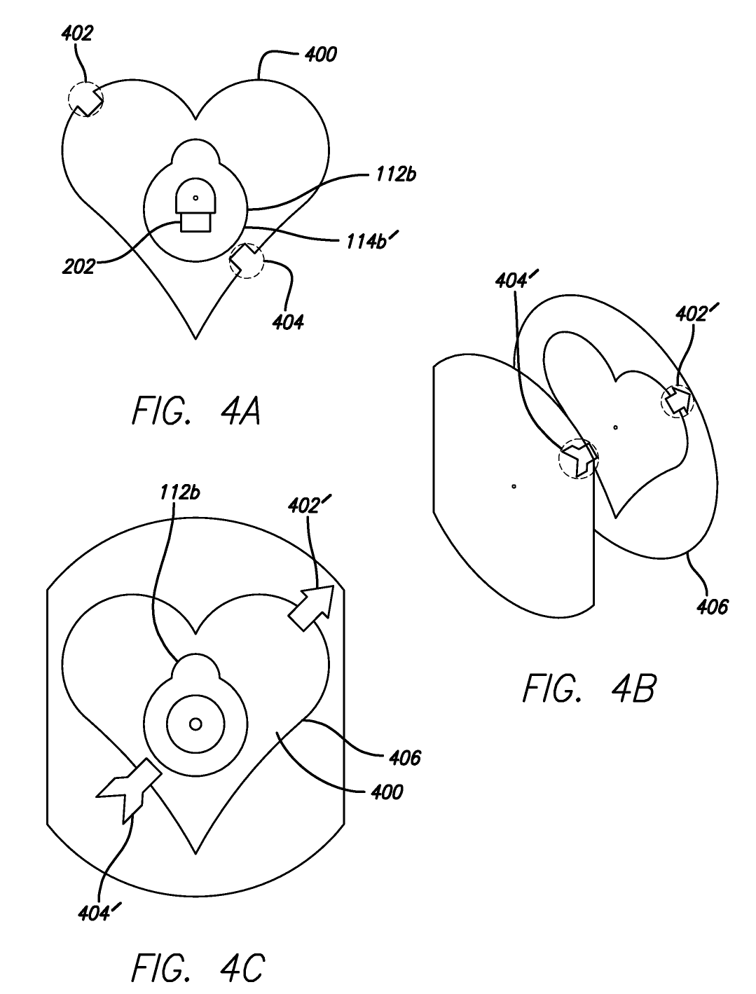

FIGS. 4A-4C are exemplary illustrations an insertion system that includes a guide tool being used in conjunction with multiple locating structures to precisely locate an insertion site for a sensor, in accordance with one embodiment of the present invention. FIG. 4A is intended to show the view from the bottom of the insertion tool 112b and footprint 114b′ and a guide tool 400 looking toward the top. For illustrations sake, the sensor 202 is shown roughly in the center of the insertion tool 112b. In this embodiment the guide tool 400 includes secondary locating structures 402 and 404. FIG. 4B illustrates an exemplary guide layer 406 that would help ensure proper alignment between the insertion tool and guide tool 400 and the guide layer 406. In some embodiments corresponding secondary locating structure 402′ is an arrow head while corresponding secondary locating structure 404′ is the tail and fletchings of an arrow. The illustration in FIG. 4C demonstrates what becomes visible from the top view looking down when the guide layer having corresponding secondary locating structures 402′ and 404′ are properly aligned with the guide tool 400 that contains the insertion tool. The illustration of the arrow through the heart is intended to demonstrate tying physical structures such as the notches in the modified footprint to the arrow shaft on the guide layer. Other embodiments can utilize different features to accomplish similar or the same visual effect to let the user know the needle will be placed in the desired location.

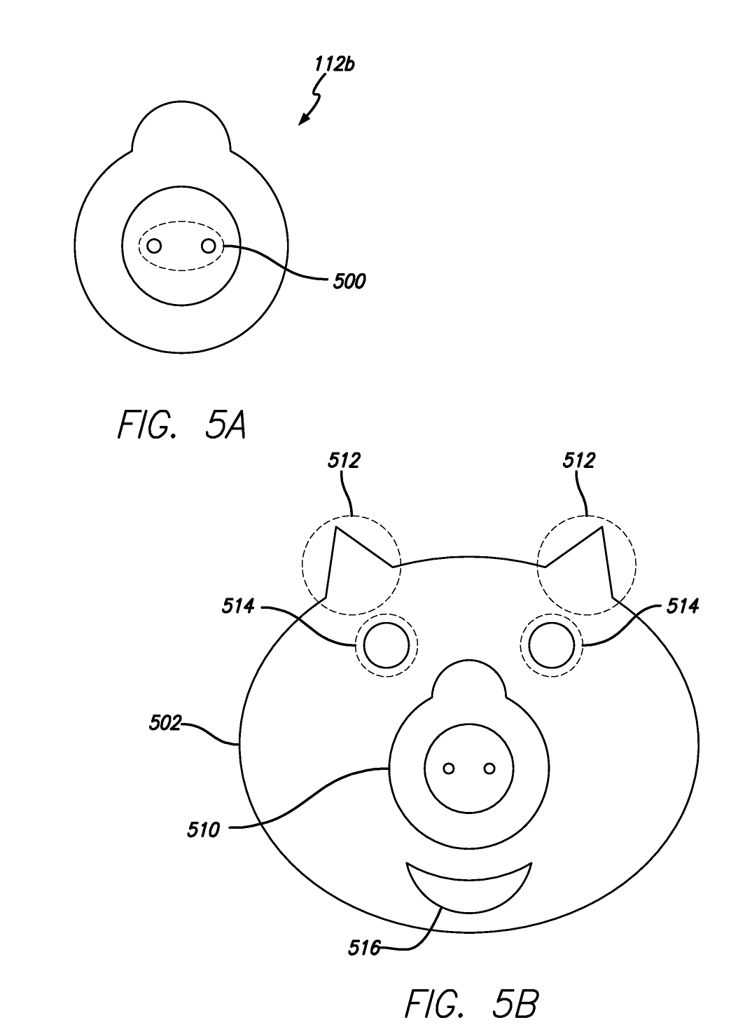

FIGS. 5A and 5B further illustration another embodiment where a first locating structure is used in conjunction with a guide layer and the insertion tool to create a cohesive structure, in accordance with one embodiment of the present invention. FIG. 5A shows a top view of the insertion tool 112b. In embodiments where the insertion tool 112b is injection molded, structures 500 can be molded into the insertion tool 112b. In other embodiments, even injection molded embodiments, structures 500 can be printed or painted on the insertion tool 112b. FIG. 5B shows a top view of guide layer 502 with insertion tool 112b installed in first locating structure 510. The guide layer 502 can further include elements 512, 514 and 516 that when combined with the insertion tool 112 with structures 500 creates a cohesive structure that may be readily identified as a pig.

In other embodiments, the insertion tool 112b itself can be made pink, while structures 500 can be painted black. Furthermore, the guide layer 502 can be made to be a similar shade of pink as the insertion tool. In other further embodiments, the guide layer 502 can be made a shade of pink that is complimentary to the insertion tool 112b. Other embodiments, particularly for insertion tools that are taller, the cohesive structure formed between the insertion tool and the insertion guide can include rocket bodies and launch pads or other animals such as giraffes on the savanna. One benefit of creating a cohesive structure between the guide layer and the insertion tool would be to help reduce anxiety in children. Alternatively, insertion tools can be intentionally designed to correspond with specific guide layer and sold in limited numbers thereby creating a market for collectible insertion tools, potentially similar to PEZ dispensers.

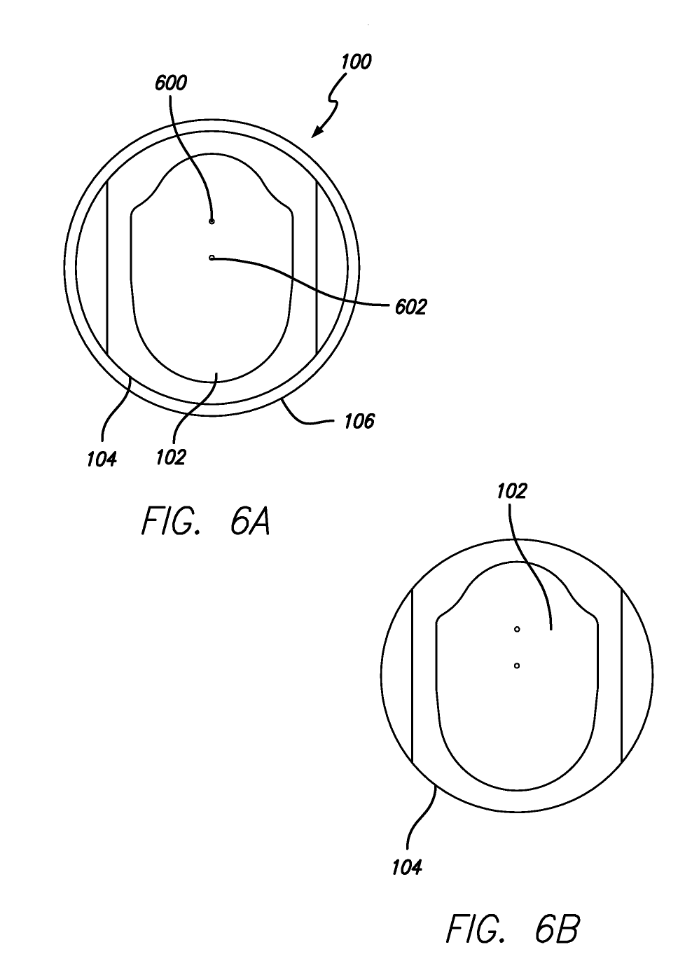

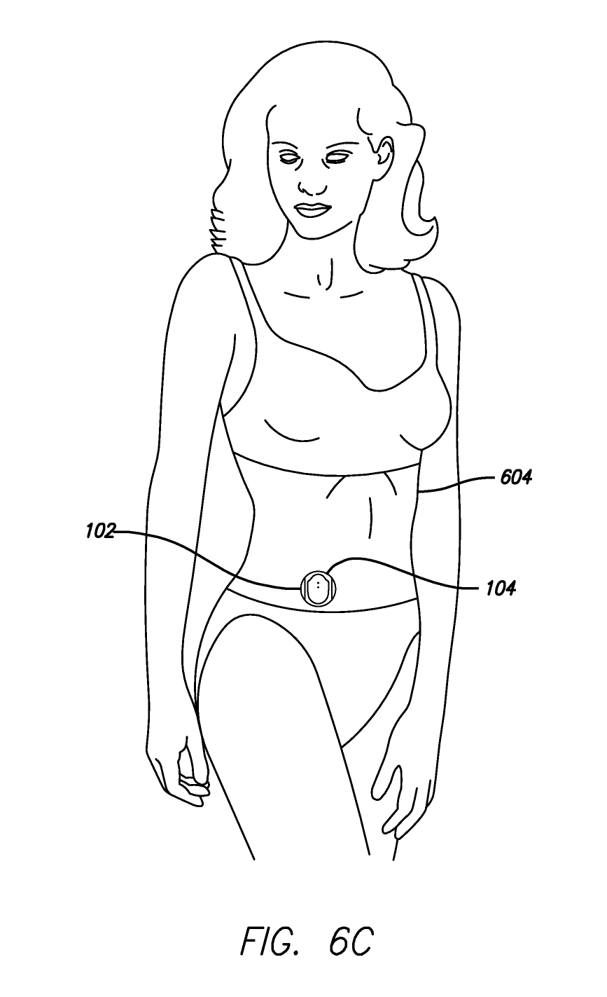

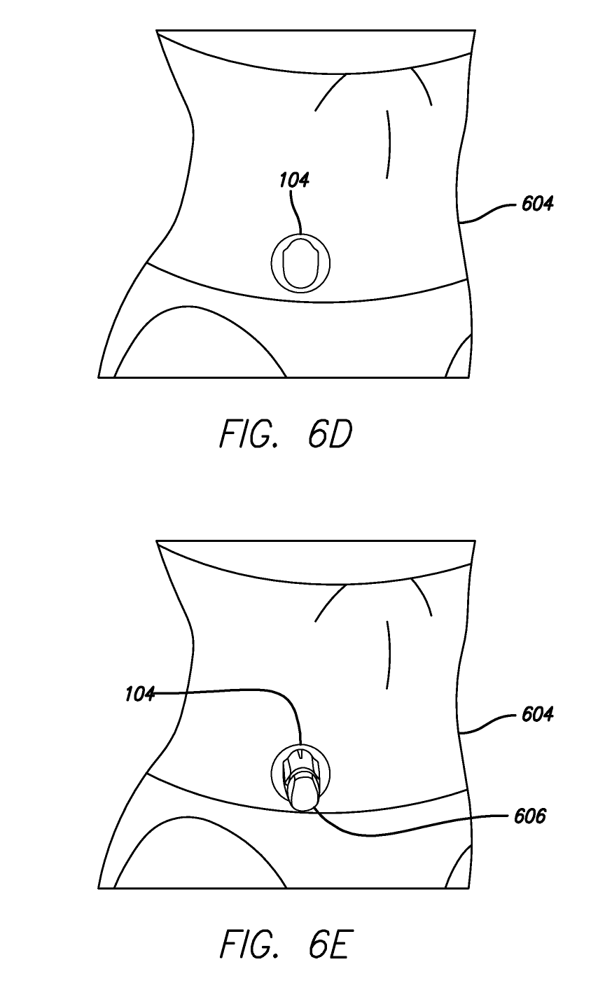

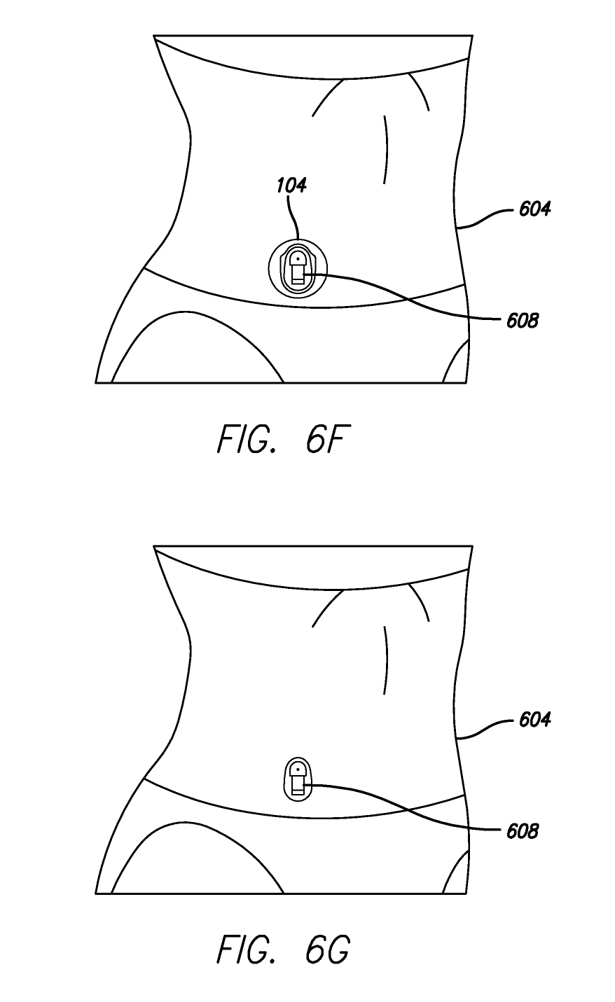

FIGS. 6A-6G illustrate various steps or operations that enable use of the insertion guide 100, in accordance with one embodiment of the present invention. FIG. 6A shows and embodiment of the insertion guide 100 that is completely intact with a protective layer 102 (clear), a guide layer 104, and the liner layer 106. Also visible are holes 600 and 602 in the protective layer 102. In some embodiments hole 602 corresponds to the insertion location for the sensor while hole 600 is a vent hole to prevent air from being trapped between the insertion guide 100 and the skin of the patient. In other embodiments, both holes 600 and 602 are vent holes that prevent air from being trapped between the insertion guide and patient. In still other embodiments, such as a dual insertion set, hole 600 correlates to a sensor location and 602 correlates to an infusion site. In still other embodiments, only a single hole 600 or 602 is made in the protective layer corresponding to the insertion site while in further embodiments, three or more holes are made in the protective layer.

The first step of using the insertion guide 100 is to remove the liner layer 106 thereby exposing the medical grade repositionable adhesive on the bottom of the guide layer 104. FIG. 6B shows the result of removal of the liner layer as only the protective layer 102 and the guide layer 104 remain. FIG. 6C is an illustration showing the second step of using the insertion guide, choosing an insertion location. In FIG. 6C an insertion location has been chosen and the guide layer 104 and protective layer 102 have been adhered to the torso 604. The medical grade adhesive on the bottom of the guide layer 104 is selected to also allow application and removal of the guide layer without excessive or painful pulling of skin and/or body hair. This allows a user to try various positions and orientations for the guide layer 104 and protective layer 102 when selecting an insertion site. In embodiments where holes 600 and 602 identify insertion locations, the holes 600 and 602 can assist the user in selecting insertion locations that are not too close in proximity to previous injection sites.

FIG. 6D show the guide layer 104 adhered to the torso 604 after the removal of the protection layer and FIG. 6E shows an insertion tool 606 being used with the guide layer 104 adhered to the torso 604. In some embodiments the guide layer is made from card stock with a thickness range between 0.010″ and 0.050″, with a preferred range of 0.025″±0.005″ and various locating features as previously discussed. In other embodiments the guide layer is made from medical grade foam with a thickness range between 1/32″ and ⅛″ with a preferred range of 0.050″±0.010″. FIG. 6F is an exemplary illustration of a sensor 608 being adhered to the torso 604 after the insertion tool is removed from the skin while the guide layer 104 is still adhered to the torso 604. FIG. 6G shows the sensor 608 alone on the torso 604 after removal of the guide layer 104 thereby completing insertion of the sensor with the guide layer 104.

While the description above refers to particular embodiments of the present invention, it will be understood that many modifications may be made without departing from the spirit thereof. The accompanying claims are intended to cover such modifications as would fall within the true scope and spirit of the present invention.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

What is claimed is:

1. An insertion system to precisely locate an insertion site for a sensor, the insertion system comprising:
an insertion guide having a protective layer, a guide layer, and a liner layer, the protective layer being disposed upon the guide layer, the guide layer having a first locating structure and an adhesive on a first side of the guide layer, the first side of the guide layer being disposed upon the liner layer; and
an insertion tool having a corresponding first locating structure to interface with the first locating structure,
wherein alignment of the first locating structure and corresponding first locating structure establishes placement of the insertion tool and subsequently the sensor.

2. An insertion system as described in claim 1, wherein the first locating structure is a void within the guide layer.

3. An insertion system as described in claim 2, where in the corresponding first locating structure is a footprint of the insertion tool.

4. An insertion system as described in claim 3, wherein the adhesive on the first side of the guide layer is medical grade repositionable adhesive.

5. An insertion system as described in claim 4, wherein the protective layer is composed of translucent material to enable viewing of the guide layer and the liner layer.

6. An insertion system as described in claim 5, wherein the liner layer is pre-scored to assist in separating the liner layer from the adhesive on the first side of the guide layer.

7. An insertion system as described in claim 6, wherein the guide layer is made from medical grade foam with a minimum thickness of 0.04 inches.

8. An insertion system as described in claim 5, wherein the insertion tool has a footprint and the insertion system further includes a guide tool being removeably attached to the footprint to create a modified footprint, the modified footprint being the corresponding first locating structure.

9. An insertion system as described in claim 8, wherein the first locating structure of the guide layer matches with the modified footprint.

10. An insertion system as described in claim 5, wherein a second locating structure is printed onto a top surface of the guide layer.

11. An insertion system as described in claim 10, wherein a second corresponding locating structure is printed on the insertion tool.

12. An insertion system as described in claim 10, wherein the insertion tool is composed of injection molded plastic and the second corresponding locating stricture is molded into the insertion tool.

13. An insertion system as described in claim 9, the modified footprint further including at least one second locating structure.

14. An insertion system as described in claim 13, wherein the guide layer further includes at least one corresponding second locating structure.

15. An insertion system as described in claim 14, wherein the corresponding second locating structure is printed onto the guide layer.

16. An insertion system as described in claim 15, wherein alignment of the second locating structure from the modified footprint and the corresponding second locating structure creates a cohesive and complimentary object using both the insertion tool and the guide layer.

17. An insertion system as described in claim 6, wherein the protective layer includes an insertion location for the sensor.

18. An insertion system as described in claim 17, wherein the protective layer further includes a vent hole to vent air trapped between the protective layer and the guide layer when the guide layer is adhered to a patient.

19. An insertion system as described in claim 7, wherein alignment of the first locating structure from the guide layer and the corresponding first locating structure creates a cohesive and complimentary object using both the insertion tool and the guide layer.
